(19) United States
(12) Patent Application Publication
(10) Pub. No.: US 2026/0265723 A1
(43) Pub. Date: Sep. 10, 2026

(54) GENETICALLY MODIFIED DIAZOTROPHS AND METHODS OF USING SAME FOR PRODUCING EXTRACELLULAR UREA

(71) Applicant: REGENTS OF THE UNIVERSITY OF MINNESOTA, Minneapolis, MN (US)

(72) Inventor: Brett M. Barney, Minneapolis, MN (US)

(21) Appl. No.: 19/552,119

(22) Filed: Feb. 27, 2026

Related U.S. Application Data
(60) Provisional application No. 63/767,956, filed on Mar. 6, 2025.

(51) Int. Cl. C12N 9/80; C12N 1/14; C12N 1/20; C12N 1/205; C12N 15/70

(52) U.S. Cl. CPC C12N 9/80; C12N 1/14; C12N 1/20; C12N 1/205; C12N 15/70

(57) ABSTRACT

A diazotrophic microbe is genetically modified to increase production of extracellular urea compared to an unmodified version of the diazotroph microbe. The genetically modified diazotrophic microbe may be used as a biofertilizer. Thus, the genetically modified diazotrophic microbe may be in co-culture with a second organism.

### CROSS REFERENCE TO RELATED APPLICATION

This application claims the benefit of U.S. Provisional Patent Application No. 63/767,956, filed Mar. 6, 2025, which is incorporated herein by reference in its entirety.

### GOVERNMENT FUNDING

This invention was made with government support under 2020-67019-31148 awarded by the National Institute of Food and Agriculture. The government has certain rights in the invention.

### SEQUENCE LISTING

This application contains a Sequence Listing electronically submitted via Patent Center to the United States Patent and Trademark Office as an .xml file entitled “0110_000770US01.xml” having a size of 13,580 bytes and created on Feb. 10, 2026. The information contained in the Sequence Listing is incorporated by reference herein.

### SUMMARY

This disclosure describes, in one aspect, a diazotrophic microbe genetically modified to increase production of extracellular urea compared to an unmodified version of the diazotroph microbe. In one or more embodiments, the diazotrophic microbe has a deletion or disruption of one or more genes of a urease operon.

In one or more embodiments, the diazotrophic microbe includes one or more exogenous genes.

In one or more embodiments, the diazotrophic microbe is a species of the genus Azotobacter, a species of the genus Gluconacetobacter, a species of the genus Azoarcus, a species of the genus Azospirillum, a species of the genus Herbaspirillum, a species of the genus Klebsiella, a species of the genus Pseudomonas, or a species of the genus Paenibacillus.

In another aspect, this disclosure describes a co-culture that includes a diazotrophic microbe genetically modified to increase production of extracellular urea compared to an unmodified version of the diazotroph microbe, and a second organism selected to feed on the extracellular urea provided by the diazotrophic microbe.

In one or more embodiments, the co-culture can include a culture medium.

In one or more embodiments, the co-culture involves applying the diazotrophic microbe to a crop plant.

In another aspect, this disclosure describes a biofertilizer that includes a diazotrophic microbe genetically modified to increase production of extracellular urea compared to an unmodified version of the diazotroph microbe.

In another aspect, this disclosure describes a method of increasing growth of a non-diazotrophic organism. Generally, the method includes co-culturing the non-diazotrophic organism with a diazotrophic microbe genetically modified to increase production of extracellular urea compared to an unmodified version of the diazotroph microbe.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1. Illustration of genetic strategies and pathway engineering to increase urea production in the model diazotroph Azotobacter vinelandii. The urease operon ureABC [3.5.1.5] was removed through markerless gene engineering approaches and used in strains AZBB730, AZBB802 and AZBB879 to convert urea into a terminal product. The Bacillus subtilis arginine permease rocE and arginase argI [3.5.3.1] were incorporated in place of Avin_16040 with a streptomycin selection marker in strains AZBB802, AZBB874 and AZBB879 to construct a complete urea cycle. The Escherichia coli arginine-insensitive acetylglutamate kinase argB [2.7.2.8] was incorporated in place of Avin_14720 with a kanamycin selection marker in strains AZBB874 and AZBB879 to circumvent arginine feedback inhibition. Each of these strategies was selected to increase the biosynthesis of urea as a terminal product in a diazotroph.

FIG. 2. Urea accumulation for various strains of Azotobacter vinelandii constructed for this study. Strain AZBB730 contained the ureABC urease operon deletion. Strain AZBB802 was constructed from strain AZBB730, and further incorporated the argB arginase and rocE arginine permease. Strain AZBB879 was constructed from AZBB802 and contained the argB acetylglutamate kinase. All strains were constructed from the parent strain AZBB721, which contains markerless deletions in the polyhydroxybutyrate biosynthesis and the alginate biosynthesis pathways to limit competition with carbon storage pathways. All strains were grown aerobically under diazotrophic growth conditions with no nitrogen provided in the liquid medium, such that all the urea produced was derived from atmospheric nitrogen. All results are based on at least four samples (n=4) and error bars represent the standard deviation.

FIG. 3. Potential of strains constructed in this study to produce sufficient extracellular nitrogen in the form of urea to support the growth of an associated alga. The algae Chlorella sorokiniana was spotted together with various Azotobacter vinelandii strains on a freshwater agar plate supplemented with sucrose but devoid of any nitrogen source in the medium. Cells were grown on a light table for two weeks. Strain AZBB730 resulted in minimal growth, while AZBB802 showed improved growth of the alga. Strain AZBB874, which is analogous to AZBB879 and produced similar levels of urea in liquid culture, showed the highest degree of algal growth, indicating that the urea levels produced from these strains were sufficient to support the growth of an associated non-diazotrophic plant surrogate.

FIG. 4. Potential of strains to produce sufficient extracellular nitrogen in the form of urea or ammonium to support the growth of an associated alga in liquid culture. The algae Chlorella sorokiniana was cultured in Erlenmeyer flasks together with various Azotobacter vinelandii strains in a freshwater/Burk's medium with sucrose but devoid of any nitrogen source in the medium. Strain DJ is the A. vinelandii strain with no modifications (wild-type). Strain AZBB679 is a previously described (Barney and Dietz, Microbial Biotechnology 17: e14523) extracellular ammonium optimized strain. Strain AZBB879 is the optimized extracellular urea strain constructed for this study. (A) Algal cells were counted using a hemacytometer over the course of a week (plotted on a Log₂-based scale). (B) At the end of one week, cultures were photographed to compare algal cell mass obtained from each culture.

### DETAILED DESCRIPTION OF ILLUSTRATIVE EMBODIMENTS

This disclosure describes diazotrophs engineered to produce urea as a terminal product, methods of engineering the diazotrophs, and methods of using the engineered diazotrophs. The engineered diazotrophs are able to fix atmospheric nitrogen and serve as a biofertilizer for other organisms in co-culture.

The cost associated with industrial nitrogen fertilizer production limits crop productivity in much of the developing world, and represents a large financial burden to farmers across the globe. Additionally, there are large energy costs associated with producing industrial fertilizers, as the Haber-Bosch process requires high pressure and temperature to convert atmospheric nitrogen into ammonia, and is responsible for one percent to two percent of all energy consumption worldwide. While some plant species have developed intricate symbiotic relationships with nitrogen-fixing (diazotrophic) microbes, allowing them to share resources and drive biological nitrogen fixation (BNF), many agricultural crops lack these intricate associations. These crops instead get their nitrogen from the soils surrounding their roots by transporting nitrogen produced by diazotrophs living in the rhizosphere. In return, the plants provide reduced carbon compounds in the form of sugars and organic acids through their root exudates that support the broad variety of microbes living near these roots that make up part of the plant microbiome.

BNF is an energetically costly process, requiring a minimum of 16 ATP, 8 protons, and 8 electrons (provided through reduced electron carriers) to fix a single N₂ nitrogen molecule into two ammonia (NH₃) molecules. The process is further complicated by the requirements for a large repertoire of proteins involved in cofactor assembly and the susceptibility of the metallo-cofactors found in these enzymes to oxygen, which can destroy the cofactors and render the enzyme useless. Because of this high metabolic cost, BNF tends to be tightly regulated inside the cell to ensure that the microbe does not invest too many of its resources to this specific process unless it is absolutely required.

Previous strategies to enhance the levels of nitrogen that are produced in various diazotrophs as a means of enhancing the biofertilizer potential of the microbes have focused on deregulation of BNF or engineered strategies to limit incorporation of ammonium into key uptake pathways and, as a result, release ammonium ions into the surrounding extracellular space (Bali et al., 1992. Applied and Environmental Microbiology 58:1711-1718; Barney et al., 2015. Applied and Environmental Microbiology 81:4316-4328; Batista et al., 2019. Biochemical Society Transactions 47:603-614; Brewin et al., 1999. Journal of Bacteriology 181:7356-7362; Ortiz-Marquez et al., 2012. Applied and Environmental Microbiology 78:2345-2352). There are alternatives to ammonium as the carrier of fixed nitrogen, including urea, which is not as central a metabolite for most diazotrophs.

The ability to produce elevated extracellular urea in the model diazotroph Azotobacter vinelandii has been demonstrated (Barney et al., 2015. Applied and Environmental Microbiology 81:4316-4328), but levels of accumulation were insufficient to have a meaningful effect on the growth of co-cultured non-diazotrophs (including plants and algae) versus what was possible through ammonium. This disclosure describes an approach that increased the levels of urea that could be obtained under aerobic diazotrophic growth conditions by a factor of 43-fold over prior strains, and nearly 350-fold higher than what is observed in wild-type A. vinelandii. In contrast to ammonium, the diazotroph is engineered to produce urea as a terminal nitrogen product, a feature that renders this approach more stable than efforts that direct fixed nitrogen to ammonium.

While described herein in the context of A. vinelandii as a model engineered diazotroph, the engineering approach, engineered diazotrophs, and methods described herein can involve the use of other diazotrophs. Exemplary alternative diazotrophs include, but are not limited to, species of the genera Gluconacetobacter, Azoarcus, Azospirillum, Herbaspirillum, Klebsiella, Pseudomonas, or Paenibacillus.

### Initial Strain Constructs

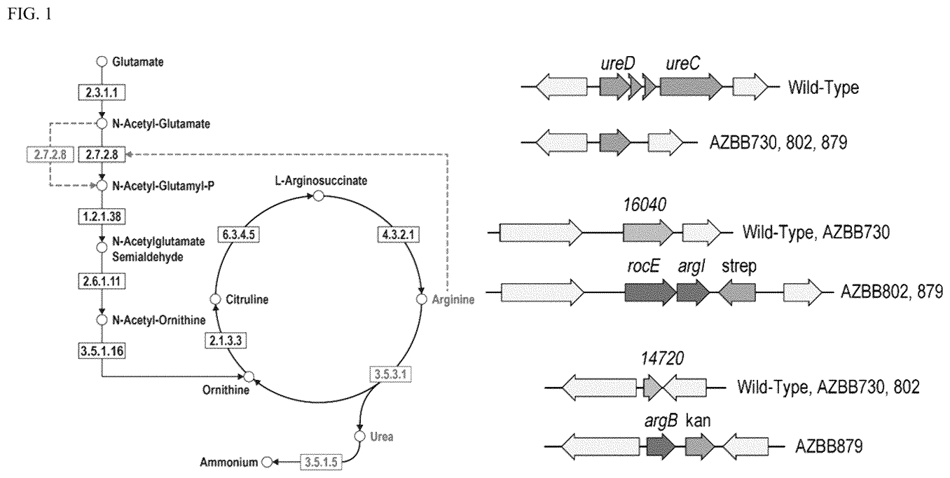

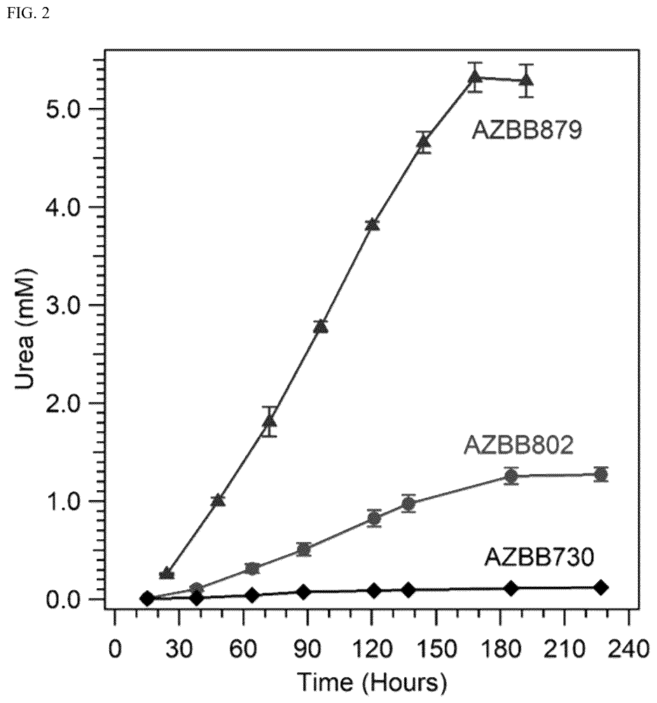

As a starting point for these studies, an A. vinelandii strain lacking the genes for polyhydroxybutyrate biosynthesis and alginate synthesis was constructed by targeting the phbBAC and algD.8.44.KJGXLIVFA operons, respectively, using markerless deletion, resulting in strain AZBB721. Following construction of AZBB721, the urease [E.C. 3.5.1.5] ureABC operon (FIG. 1) was targeted for deletion using the conditionally toxic SacB gene strategy for counter-selection (Dietz et al., 2024. Microbiology Spectrum 12 (1), e02478-23), resulting in the deletion of the urease operon in a strain lacking any antibiotic markers, to obtain strain AZBB730. This strain served as the baseline strain for urea production, and was cultured in Burk's medium in the absence of any fixed nitrogen source to force the strain to grow diazotrophically while monitoring urea accumulation in the culture medium. Extracellular urea concentrations for AZBB730 achieved levels of ~120 M (FIG. 2) following several days of growth. Strain AZBB730 served as the parent strain for the remaining modifications to further increase urea production.

Incorporating a foreign arginase into A. vinelandii to convert intracellular arginine into urea Arginase genes from Azospirillum brasilense, Rhodobacter sphaeroides and Bacillus subtilis were selected and cloned, but a further increase in urea accumulation was observed only for strains containing the arginase ArgI from B. subtilis. The argI arginase from B. subtilis was cloned either as a single gene or in combination with the upstream rocE gene encoding an arginine/ornithine permease. The strain AZBB802 containing both the rocE and argI genes behind the highly transcribed S-layer protein (Avin_16040) expression system (Arriola et al., 2017. Plant J 93:566-586; Barney and Plunkett, 2022. Appl Microbiol Biotechnol 106:5051-5061; Barney et al., 2017. Appl Environ Microbiol 83: e01534-01517) resulted in the highest increases in urea, achieving extracellular concentrations of ~1.2 mM, a ten-fold improvement over AZBB730 (FIG. 2). A strain lacking the rocE gene (AZBB833) produced about half as much urea.

While described herein in the context of an exemplary embodiment in which the diazotroph is genetically modified to express the arginase from B. subtilis, the genetically modified diazotrophs and methods described herein can involve the use of any arginase that is expressible in the diazotroph being modified. For example, the arginases from Rhodobacter sphaeroides and from Azospirillum brasilense were selected because these microbes are diazotrophs, but they did not produce urea when used to modify A. vinelandii, perhaps because these are remnants of genes that have not been used for some time. In contrast, B. subtilis ArgI is well studied and similar enzymes from similar species—e.g., Bacillus spp. and/or Paenibacillus spp.—have similar activities. That the arginase from B. subtilis works as well as it does in Azotobacter was a surprise because the active site of the B. subtilis arginase contains a manganese metal coordination site. The data of FIG. 2 show, however, that B. subtilis ArgI arginase increased extracellular urea production without manganese added to the growth medium. When the growth medium was supplemented with manganese, no significant improvement was observed.

### Increasing Intracellular Levels of Arginine to Drive Further Urea Production

In pilot experiments where extraneous arginine was supplemented into the growth medium, strain AZBB802 increased urea accumulation (data not shown). These results indicated that urea accumulation was limited in A. vinelandii by available arginine substrate for the heterologous arginase ArgI. As the overall goal was to accumulate urea from nitrogen that is fixed through BNF by the enzyme nitrogenase, AZBB802 may be able to further increase urea production by increasing the intracellular concentrations of arginine. In most microbes, arginine biosynthesis is feedback inhibited early in the arginine biosynthesis pathway at the point of N-acetylglutamate kinase encoded by argB (FIG. 1).

To overcome this potential feedback inhibition in A. vinelandii, the arginine-insensitive argB gene from E. coli (Ramón-Maiques et al., 2006. Journal of Molecular Biology 356:695-713; Ramón-Maiques et al., 2002. Structure 10:329-342) was incorporated. The E. coli ArgB lacks an arginine binding site that is found in many strains, including pseudomonads and corynebacteria (Ramón-Maiques et al., 2006. Journal of Molecular Biology 356:695-713). By expressing the E. coli argB gene behind the promoter of the conserved hypothetical protein (Avin_14720), urea accumulation was further increased (FIG. 2, strain AZBB879) another four-fold over AZBB802 to ~5.3 mM. These results are dramatically higher than AZBB730 and wild-type A. vinelandii.

While described herein in the context of an exemplary embodiment in which the diazotroph is genetically modified to include the arginine-insensitive argB gene from E. coli, the genetically modified diazotrophs and methods described herein can involve use of other enzymes having similarities to argB in Azotobacter, Pseudomonas, Corynebacterium, etc. Based on the crystal structures of the enzyme, one can target specific residues of the arginine binding site for mutation, thereby blocking arginine inhibition of the enzyme. Such an approach has been used in Corynebacterium and is the way most arginine dietary supplement products are produced. Alternatively, one can use any argB enzyme that lacks an arginine binding site. The E. coli argB enzyme (and similar enzymes from other species, e.g., Salmonella) is truncated and lacks the inhibition site altogether. Thus, any enzyme that is similar in primary protein structure—e.g., truncated for the start of the protein sequence with similarity to the E. coli enzyme—can be used, although yields may vary.

Also, while described herein in the context of exemplary embodiments in which exogenous gene expression (e.g., E. coli argB, B. subtilis argI, etc.) is regulated by the Avin 14720 or Avin_16040, the genetically modified diazotrophs and methods described herein can involve the use of any promoter that results in sufficient expression of the exogenous gene in Azotobacter.

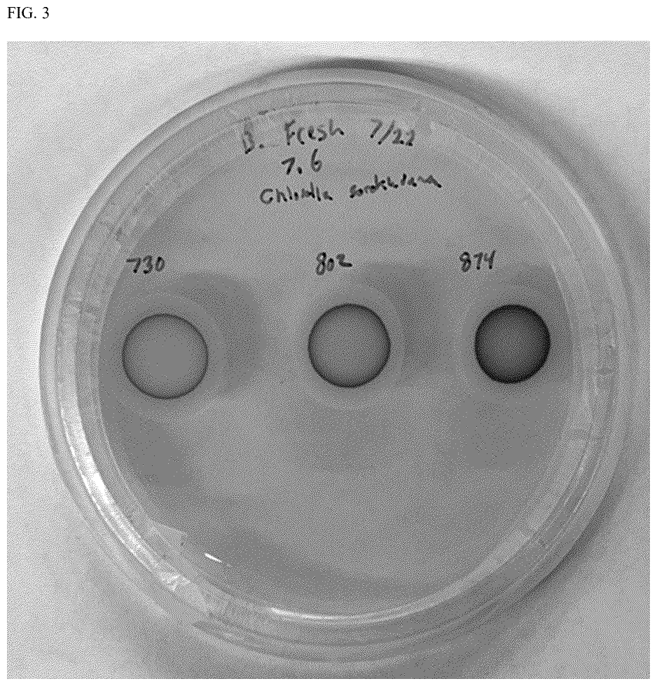

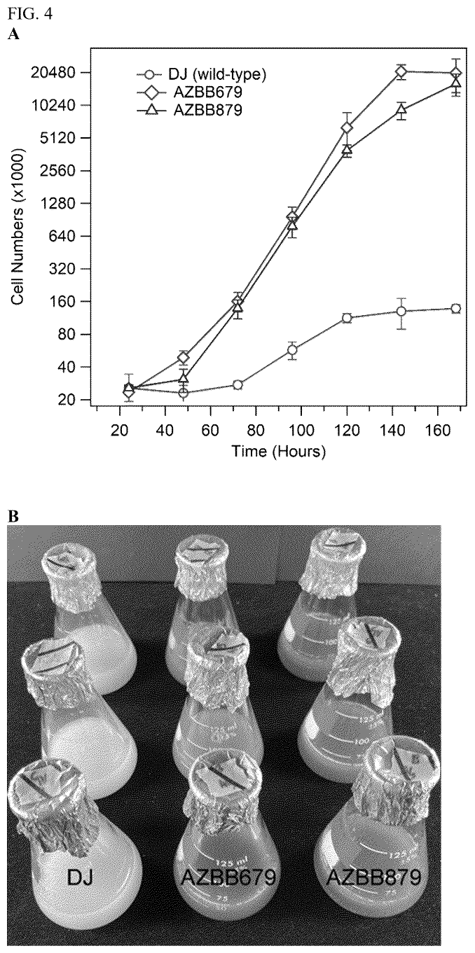

Strains AZBB730, AZBB802 and AZBB879 were all grown diazotrophically while measuring initial growth rates, and resulted in doubling times of 3.3 hours for AZBB730, 3.3 hours for AZBB802 and 3.4 hours for AZBB879, which is similar to what was found for the wild-type (DJ) strain. This indicates that these strains with an extracellular urea sink did not incur a large burden in terms of BNF. In addition, no spontaneous revertants or extragenic suppressors (i.e., “cheaters”) were observed in multiple independent experiments with multiple replicates. Yields of urea were steady across the course of seven days, and unlike prior approaches that focused on producing high levels of ammonium, no evidence of strain instability—e.g., manifesting as lower urea yields for a specific sample—was observed. As a further measure of the potential to provide urea to its surroundings, strains AZBB730, AZBB802, and AZBB874 were co-cultured with the non-diazotrophic phototrophic alga C. sorokiniana (FIG. 3 and FIG. 4). Consistent with the data in FIG. 2, the increased amounts of extracellular nitrogen were sufficient to support the growth of a non-diazotrophic phototroph both on solid agar plates and in liquid culture.

### Rationale for Urea as the Target for Nitrogen Delivery by Biofertilizer Strains

There have been many approaches to engineer nitrogen-fixing microbes such as A. vinelandii to produce elevated levels of extracellular nitrogen compounds to serve a potential role in enhanced biofertilizer applications. The overwhelming majority of these approaches target ammonium production. Ammonia is the product of nitrogenase, the enzyme that converts atmospheric nitrogen into fixed nitrogen. Genetic manipulation strategies that target elevated ammonium accumulation all struggle to some degree because biological nitrogen fixation (BNF) is tightly regulated in most diazotrophs.

Often, when targeting ammonium accumulation, modifications to circumvent these regulatory processes can be quite extensive. Additionally, because ammonium is a central nitrogen metabolite, there is minimal incentive for cells to continue to produce ammonium once they find themselves in an environment with elevated levels of ammonium, especially when the continual stress of elevated BNF comes at a fitness cost to the microbe. This fundamental feature of ammonium means that strategies to deregulate BNF are prone to evolving spontaneous mutations (i.e., “cheaters”) that can disrupt any of the myriad enzymes involved in biological nitrogen fixation, resulting in strain instability as cultures develop populations that take advantage of the high ammonium that is available to them.

Urea, in contrast to ammonium, is not a central metabolite in most diazotrophs. While many diazotrophs will use urea by producing two ammonium molecules per molecule of urea through the action of urease (FIG. 1), the urea itself does not generally deregulate BNF (Knutson et al., 2018. Appl Microbiol Biotechnol 102:10315-10325). Additionally, urea can be turned into a terminal product by simply eliminating the urease from a microbe's enzymatic arsenal (Barney et al., 2015. Applied and Environmental Microbiology 81:4316-4328). These two aspects of urea have been the primary driver for the approach described herein, in which urea is targeted as a way of delivering reduced nitrogen from diazotrophic microbes to plant or algal hosts. Once urease has been deleted from the diazotrophic microbe, the requirement of that microbe to evolve a new process to utilize elevated urea levels or obtain a full urease gene from the environment through horizontal gene transfer would represent a large evolutionary barrier. In a large population where microbes could evolve spontaneous mutations that shut down BNF, these mutants would still be unable to capitalize on the elevated urea due to a lack of the ability to metabolize it in the parent strain.

While described herein in the context of exemplary embodiments in which urease operon ureABC [3.5.1.5] is deleted, the engineering approach, engineered diazotrophs, and methods described herein can involve any mutations and/or engineering that reduces conversion of urea to ammonium so that the diazotroph accumulates urea, even if the urea accumulation is less than exhibited in the strains described herein. For example, mutations that reduce the activity of the urease operon ureABC [3.5.1.5] sufficiently that the diazotroph is able to accumulate urea are possible. In such embodiments, additional strategies may need to be employed to ensure that the modified diazotrophs do not simply evolve back to a fully functional urease operon. Alternatively, strategies that produce intracellular metabolites that inhibit the urease enzyme may involve ensuring that the metabolites do not also target other enzymes.

Overcoming Barriers to Urea Production in A. vinelandii

There are several common metabolic pathways that could increase intracellular urea concentrations, but one of the most promising is that of the urea cycle from arginine biosynthesis. While this pathway is common in many higher animals as a means of removing excess nitrogen as a waste product, most diazotrophs lack the enzyme arginase that produces urea and ornithine from arginine. A. vinelandii also lacks a known arginase (ArgI) enzyme (Setubal et al., 2009. Journal of Bacteriology 191:4534-4545). Successful biosynthesis of urea through arginase requires identifying a suitable enzyme that is active in the microbial host, and finding suitable levels of expression of that enzyme. Three different arginase systems were initially selected for testing: B. subtilis, Rhodobacter sphaeroides and Azospirillum brasilense. Of these, only the arginase from B. subtilis yielded increases in urea when expressed in the S-layer protein expression system of A. vinelandii. The arginases from R. sphaeroides and A. brasilense did not increase urea yield and were abandoned.

Additionally, while increased urea production was observed with the arginase alone, constructs that included the upstream arginine permease gene from B. subtilis in addition to the arginase resulted in further increased levels of urea, while constructs containing the arginase alone only achieved about half as much urea when the arginine permease was included. Further efforts to incorporate alternative RBS sites upstream of the arginase provided similar levels of urea, indicating that further engineering to increase arginase function would likely have a minimal role in further urea enhancement. The effort to produce arginase through this strategy resulted in a ten-fold increase in urea accumulation for the AZBB802 strain over the AZBB730 strain that only contains the disruption in the urease. These results demonstrate that engineering further urea production by constructing a complete urea cycle in a diazotroph such as, for example, A. vinelandii is a viable strategy for improving the biofertilizer potential of this strain.

### Deregulation of Arginine Biosynthesis to Provide Substrate to Arginase

An additional metabolic pathway that may be engineered to increase urea accumulation is the arginine biosynthesis pathway. A. vinelandii contains an N-acetylglutamate kinase (argB) that is expected to be feedback inhibited by arginine in the cell. In many commercial strains targeted for the production of arginine, specific mutations that disrupt the arginine binding site of argB are a specific target for increasing arginine biosynthesis. A simpler strategy for use in diazotrophs incorporated the arginine-insensitive argB from E. coli, which lacks an arginine binding site and remains active in the presence of increased arginine. The argB gene from E. coli was cloned under the control of the constitutive promoter of the conserved hypothetical gene (Avin_14720) to overcome the feedback inhibition of arginine in the indigenous argB of A. vinelandii. When combined with the arginase in strain AZBB802, a further four-fold increase in urea production was achieved, indicating that the ArgB of A. vinelandii is inhibited by arginine, and as a result, was limiting the production of urea in AZBB802 by failing to provide adequate arginine to the arginase. These two additions to the urease deletion strain of A. vinelandii resulted in a significant and stepwise increase in urea production, demonstrating that urea can be a viable target for strategies to increase the biofertilizer potential of a diazotroph.

### Comparison Between Urea and Ammonium Accumulation Strategies

Prior efforts to engineer the model diazotroph A. vinelandii by deleting or inactivating urease resulted in elevated levels of urea accumulating the extracellular environment, but these levels were too low to provide meaningful levels of urea to a potential plant or algal host. Prior methods of producing extracellular ammonium yielded ammonium concentrations greater than 25 mM (Plunkett et al., 2020. Microbial Cell Factories 19:107; Barney et al., 2022. Applied Microbiology and Biotechnology 106:5051-5061; Barney et al., 2024. Microbial Biotechnology 17). However, these strains are susceptible to the development of a cheater (extragenic suppressor) population and the elevated ammonium levels are known to inhibit further nitrogen production, even in strains where BNF has been partially deregulated (Plunkett et al., 2020. Microbial Cell Factories 19:107; Barney et al., 2022. Applied Microbiology and Biotechnology 106:5051-5061; Barney et al., 2024. Microbial Biotechnology 17; Knutson et al., 2018. Appl Microbiol Biotechnol 102:10315-10325).

The enhanced urea production strain AZBB879 described herein accumulated over 5 mM of urea. Since urea contains two equivalents of nitrogen per molecule, this urea accumulation is equivalent to 10 mM of ammonium. In this manner, the strains achieved urea concentrations along the same order as ammonium, but with no manipulation to the regulation of BNF, resulting in a strain with minimal growth inhibition. Additionally, because the urea is a terminal product, the strain is less susceptible to the loss of this nitrogen to subpopulations that develop spontaneous mutations.

Thus, this disclosure illustrates the potential to target urea production in a diazotrophic microbe as an alternative to ammonium, resulting in a strain with improved stability. Urea is already used in many parts of the world as a fertilizer for agriculture, where it is preferred for foliar applications because it is rapidly and efficiently absorbed by leaves. In addition to potential applications where A. vinelandii could be applied to soils as a potential biofertilizer strain, the stable production of this nitrogen product could be amenable to scale-up in fermentations to produce urea onsite using sugars or other agricultural byproducts. The dramatic improvements demonstrated in this work provide a proof of concept to transfer this approach to other diazotrophs. By removing endogenous ureases from other strains in microbial consortia, the approach could be used to optimize delivery of the nitrogen fixed through BNF to the plant in the form of urea.

Thus, this disclosure describes, in one aspect, a diazotrophic microbe genetically modified to produce extracellular urea in an amount greater than a comparable control diazotrophic microbe. As used herein, a “diazotroph” and “diazotrophic microbe” are used interchangeably to refer to a microbe capable of fixing nitrogen from the atmosphere into a usable form of nitrogen, such as urea. Diazotrophs can be naturally found free-living or in association with other organisms, such as plants. Free-living diazotrophs typically live in the soil and secrete fixed nitrogen into the surrounding soil. Plants living in the soil can acquire and use this nitrogen. However, nitrogen secreted into the soil is susceptible to loss and may be inconsistently used by plants.

Diazotrophs that live within or in associate with other organisms without causing apparent disease are typically referred to as endophytes. Some endophytes benefit an associated organism by transferring nitrogen to the associated organisms with greater efficiency than the nitrogen can be transferred by a free-living diazotroph. In one or more embodiments, the microbe described herein is an endophyte.

As used herein, a “comparable control diazotrophic microbe” refers to a diazotrophic microbe lacking the genetic modifications that allow the modified microbe to produce urea as a metabolic terminal end product. One example of a comparable control diazotrophic microbe is a naturally occurring, wild-type version of the diazotrophic microbe.

In one aspect, this disclosure describes co-cultures and co-culture methods that involve a diazotroph microbe genetically modified to produce extracellular urea and a second organism selected to feed on the extracellular urea produced by the diazotroph. In one or more embodiments, the second organism can be a non-diazotroph. In one or more embodiments, the co-culture may be an in vitro culture in which the genetically modified diazotroph and the non-diazotroph second organism are grown in or on culture medium. The culture medium may be liquid or solid.

In one or more embodiments, the co-culture may be ex vitro—e.g., the genetically modified diazotroph may be applied to, on, or into an environment in which the second organism is growing. The second organism may be cultivated or native to the environment. In this context, the genetically modified diazotroph may be a biofertilizer, providing extracellular urea to the non-diazotroph and, consequently, increase the growth rate of the non-diazotroph.

Biofertilizers provide a range of potential benefits versus current industrial nitrogen production routes. In situ biofertilizer production could displace and circumvent transportation costs and associated environmental effects related to producing and distributing Haber-Bosch-derived industrial fertilizers. Biological assimilation of nutrients and/or timed release of urea can mitigate issues associated with, for example, agricultural residue runoff from excessive application of industrial fertilizers, leading to eutrophication of nearby water supplies and streams.

In one or more embodiments, the genetically modified diazotrophic microbe can be derived from Azotobacter vinelandii. In one or more embodiments, the genetically modified diazotroph may be derived from any species of the genus Gluconacetobacter, any species of the genus Azoarcus, any species of the genus Azospirillum, any species of the genus Herbaspirillum, any species of the genus Klebsiella, any species of the genus Pseudomonas, or any species of the genus Paenibacillus. As used herein, “derived from” in connection with a microbe simply allows for the host cell to possess one or more genetic modifications before being modified to excrete a nitrogen-containing compound.

In one or more embodiments, the second organism may be selected to be able to feed on the extracellular urea produced by the diazotroph and to produce a product of interest. Current biofuel feedstock crops such as corn for ethanol require substantial amounts of nitrogen inputs. Potential future production of biomass using next generation feedstocks such as algae provide significant improvements in overall yield that could be orders of magnitude higher than current conventional land plant crops. Since current nitrogen requirements for the growth of biofuel crops are derived from energy intensive industrial processes such as Haber-Bosch, the energy use efficiency of current biofuel crops has been questioned. A significant amount of the energy acquired from, for example, corn ethanol, soybean biodiesel, and/or next generation biofuel crops (e.g., algae) may need to be diverted back to these industrial processes to supply the energy required for additional industrial nitrogen fixation. Improvements in final biomass yield can involve concomitant increases in macronutrient inputs such as nitrogen.

In one or more embodiments wherein the genetically modified diazotroph is an endophyte, the associated second organism is a plant. The plant may be a vascular plant, such as a legume, tree, or grass. The plant may alternately be a photosynthetic organism, such as algae. The associated organism may be a biofuel feed crop, such as corn, wheat, soy, algae, sugarcane, sugar beet, sorghum, grasses, or canola. In one or more embodiments, the associated plant may be a crop (e.g., a primary crop) that does not naturally associate with Rhizobium (non-nodules). In one or more of these embodiments, the crop plant is a non-legume plant. Exemplary crop plants include, but are not limited to, corn, wheat, rice, barley, sugar beet, etc. The plant may be grown on any suitable scale and using any suitable method, such as soil-based growth, hydroponic growth, or any other growing system.

In one or more embodiments, the microbe may not require formation of a specialized structure, such as a root nodule, to grow symbiotically with an organism. Some diazotrophic microbes grow within the root nodules of other organisms, such as plants. However, many plants and other organisms that require nitrogen do not form root nodules or other structures to promote colonization by diazotrophic microbes. Microbes that can grow in co-culture with other plants without specialized structures may be more adaptable to co-culture with plants that may not otherwise be colonized by diazotrophic microbes.

In another aspect, the present disclosure describes a method of increasing growth of an organism in co-culture with a genetically modified endophytic diazotrophic microbe. Generally, this method includes co-culturing an organism with a genetically modified endophytic diazotrophic microbe. The genetically modified endophytic diazotrophic microbe may be consistent with any described herein. In one or more embodiments, the genetically modified diazotrophic microbe is within the organism in co-culture. For example, if the organism in co-culture is a flowering plant, the microbe may be within the leaves, roots, stem, and/or flowers of the plant.

In one or more embodiments, the second organism in co-culture is not a diazotroph. The organism may be a photosynthetic organism, such as a plant or an alga. The plant may be a vascular plant, such as a legume, tree, or grass. The organism may be a biofuel feed crop, such as corn, soy, algae, sugarcane, sugar beet, sorghum, or canola.

In one or more embodiments, increasing the growth of the organism includes increasing the growth rate of the organism. In one or more embodiments, increasing the growth of the organism includes increasing the cell density of the organism. In one or more embodiments, increasing the growth of the organism includes increasing the crop yield of the organism. In one or more embodiments, increasing the growth of the organism may be a result of increased nitrogen availability.

While endophytes typically have a symbiotic relationship with co-cultured organisms, microbes can cause an immune response in co-cultured plants. For example, a plant may produce antibiotic compounds in response to detecting certain microbial metabolites. For the co-cultured plant, production of an immune response can be energetically costly and may divert resources away from growth. Thus, to increase growth of co-cultured plants, genes involved in producing immunogenic compounds within the diazotrophic microbe may be disrupted or deleted.

In the preceding description and following claims, the term “and/or” means one or all of the listed elements or a combination of any two or more of the listed elements; the terms “comprises,” “comprising,” and variations thereof are to be construed as open ended—i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

As used herein, “have,” “has,” “having,” “include,” “includes,” “including,” “comprise,” “comprises,” “comprising” or the like are used in their open-ended inclusive sense, and generally mean “include, but not limited to,” “includes, but not limited to,” or “including, but not limited to.” Further, wherever embodiments are described herein with the language “have,” “has,” “having,” “include,” “includes,” “including,” “comprise,” “comprises,” “comprising” and the like, otherwise analogous embodiments described in terms of “consisting of” and/or “consisting essentially of” are also provided. The term “consisting of” means including, and limited to, that which follows the phrase “consisting of.” That is, “consisting of” indicates that the listed elements are required or mandatory, and that no other elements may be present. The term “consisting essentially of” indicates that any elements listed after the phrase are included, and that other elements than those listed may be included provided that those elements do not interfere with or contribute to the activity or action specified in the disclosure for the listed elements.

As used herein, the word “exemplary” means to serve as an illustrative example and should not be construed as preferred or advantageous over other embodiments.

As used herein, the terms “preferred” and “preferably” refer to embodiments of the invention that may afford certain benefits under certain circumstances. However, other embodiments may also be preferred under the same or other circumstances. Furthermore, the recitation of one or more preferred embodiments does not imply that other embodiments are not useful and is not intended to exclude other embodiments from the scope of the invention.

In the preceding description, particular embodiments may be described in isolation for clarity. Reference throughout this specification to “one embodiment,” “an embodiment,” “certain embodiments,” “one or more embodiments,” or “one or more embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments. Thus, features described in the context of one embodiment may be combined with features described in the context of a different embodiment except where the features are necessarily mutually exclusive.

In several places throughout the above description, guidance is provided through lists of examples, which examples can be used in various combinations. In each instance, the recited list serves only as a representative group and should not be interpreted as an exclusive list.

For any method disclosed herein that includes discrete steps, the steps may be performed in any feasible order. And, as appropriate, any combination of two or more steps may be performed simultaneously.

### EXAMPLES

The present invention is illustrated by the following examples. It is to be understood that the particular examples, materials, amounts, and procedures are to be interpreted broadly in accordance with the scope and spirit of the invention as set forth herein.

### Materials

Azotobacter vinelandii strain DJ (ATCC BAA-1303) and Bacillus subtilis strain 168 (ATCC 23857) are available from the American Type Cultures Collection (Manassas, VA). Escherichia coli MG1655 was kindly provided by Michael Sadowsky. E. coli JM109 was obtained from New England Biolabs, Inc. (Ipswich, MA). All chemicals and reagents used in this study were obtained from Fisher Scientific (Pittsburgh, PA) or Sigma-Aldrich Chemical Company (St. Louis, MO).

### Culture Growth

A. vinelandii was grown in Burk's medium at 26° C. on a shaker table at 180 rpm as previously described (Dos Santos, P C. 2011. Methods in Molecular Biology 766:81-92; Plunkett et al., 2020. Microbial Cell Factories 19:107).

### Strain Construction

All strains were constructed following previously described methods (Dos Santos, P C. 2011. Methods in Molecular Biology 766:81-92; Dietz et al., 2024. Microbiology Spectrum 12; Eberhart et al., 2016. Journal of Applied Microbiology 120:1595-1604). For manipulations that used counter-selection and SacB toxicity, strains containing the sacB gene were first grown on Burk's medium with glucose substituted for sucrose.

A list of strains used in this study are provided in Table 1. Plasmids and primers used to construct new strains are provided in Table 2 and Table 3.

TABLE 1
Strains

| | | | Parent |
|---|---|---|---|
| A. vinelandii Strain | Plasmid utilized | Genetic features | Strain/Reference |
| DJ | None | Wild-type | (1) |
| AZBB679 | | nifA behind dgoK promoter and RBS site, | (2) |
| | | ΔnifA2, ΔnifLA | |
| AZBB721 | | ΔalgD.8.44.KJGXLIVFA, ΔphbBAC | (3) |
| AZBB727 | pPCRURE90 | ureABC::lacZ-tet^{R}, ΔalgD.8.44.KJGXLIVFA, | AZBB721 |
| | | ΔphbBAC | |
| AZBB729 | pPCRURE91 | ureABC::lacZ-tet^{R} with single homologous | AZBB727 |
| | | recombination of pPCRURE92, | |
| | | ΔalgD.8.44.KJGXLIVFA, ΔphbBAC | |
| AZBB730 | | ΔureABC, ΔalgD.8.44.KJGXLIVFA, ΔphbBAC | AZBB729 |
| AZBB802 | pPCRARG42 | Avin_16040::BSU40330 (rocE, Bacillus | AZBB730 |
| | | subtilis) and BSU40320 (argI, Bacillus subtilis) | |
| | | strep^{R}, ΔureABC, ΔalgD.8.44.KJGXLIVFA, | |
| | | ΔphbBAC | |
| AZBB833 | pPCRARG50 | Avin_16040::BSU40320 (argI, Bacillus | AZBB730 |
| | | subtilis) strep^{R}, ΔureABC, | |
| | | ΔalgD.8.44.KJGXLIVFA, ΔphbBAC | |
| AZBB875 | pPCRARG88 | Avin_16040:BSU40330 (rocE, Bacillus | AZBB730 |
| | | subtilis) and BSU40320 (argI, Bacillus subtilis, | |
| | | improved RBS) strep^{R}, ΔureABC, | |
| | | ΔalgD.8.44.KJGXLIVFA, ΔphbBAC | |
| AZBB879 | pPCRARG89 | Avin_14720::argB (Escherichia coli, improved | AZBB875 |
| | | RBS), Avin_16040::BSU40330 (rocE, Bacillus | |
| | | subtilis) and BSU40320 (argI, Bacillus subtilis, | |
| | | improved RBS) strep^{R}, ΔureABC, | |
| | | ΔalgD.8.44.KJGXLIVFA, ΔphbBAC | |

(1) Setubal et al., 2009. J Bacteriol 191: 4534-4545.
(2) Barney and Dietz, 2024. Microbial biotechnology 17(7): e14523.
(3) Eberhart et al., 2016. J Appl Microbiol 120: 1595-1604.

TABLE 2
Plasmids and relevant derivatives used for the construction of Azotobacter vinelandii manipulated strains

| | | | Reference |
|---|---|---|---|
| Plasmid | Relevant Genes Cloned or Plasmid Manipulation | Parent Vector(s) | and/or Source |
| pBB284 | pUC19 based plasmid with deoxyviolacein cassette for | | (2) |
| | purple/white selection. | | |
| pBB361 | Moved deoxyviolacein cassette from pBB284 into | pBB284, | This Study |
| | pBBTET3 derivative for purple/white selection in a | pBBTET3 | |
| | tetracycline resistance based plasmid. | | |
| pBBTET3 | pUC19 derived plasmid with tetracycline resistance | | (4) |
| | marker in place of Amp. | | |
| pLACZF19 | Plasmid containing lacZ and tetracycline resistance | | (5) |
| | cassette. | | |
| pPCRARG35-2 | Cloned BSU40330 and BSU40320 genes from B. | pBBTET3 | This Study |
| | subtilis into pBBTET3 and removed internal NdeI site. | | |
| pPCRARG42 | Moved BSU40330 and BSU40320 operon behind | pPCRARG35-2, | This Study |
| | Avin_16420 gene promoter in pPCRUNKK41. | pPCRUNKK41 | |
| pPCRARG44 | Added new NdeI site at start of BSU40320 for removal | pPCRARG42 | This Study |
| | of BSU40330 gene. | | |
| pPCRARG50 | Removed BSU40330 from pPCRARG44. | pPCRARG44 | This Study |
| pPCRARG83 | Cloned argB gene from E. coli into pBB361 plasmid. | pBB361 | This Study |
| pPCRARG88 | Modified region between BSU40330 and BSU40320 | pPCRARG42 | This Study |
| | to engineer strong RBS site upstream of BSU40320. | | |
| pPCRARG89 | Moved E. coli argB gene behind Avin_14720 gene | pPCRARG83, | This Study |
| | promoter with strong RBS site and kanamycin | pPCRAZP10 | |
| | selection in pPCRAZP10 plasmid. | | |
| pPCRAZP1 | Cloned Avin_14720 gene and flanking regions from A. | pBB284 | (2) |
| | vinelandii into pBB284 plasmid. | | |
| pPCRAZP4 | Modified pPCRAZP1 and performed plasmid PCR to | pPCRAZP1 | This Study |
| | remove Avin_14720 and incorporate restriction site for | | |
| | new expression plasmid. | | |
| pPCRAZP7 | Incorporated kanamycin cassette and deoxyviolacein | pPCRKAN4, | This Study |
| | cassette into pPCRAZP4 for optimized expression | pPCRPRP7 | |
| | vector with kanamycin selection following integration | | |
| | into A. vinelandii. | | |
| pPCRAZP10 | Performed PCR on pPCRAZP7 to incorporate strong | pPCRAZP7 | This Study |
| | RBS site into Avin_14720 promoter. | | |
| pPCRKAN4 | Plasmid carrying kanamycin selection cassette. | | (7) |
| pPCRPRP7 | Plasmid carrying deoxyviolacein cassette. | | (2) |
| pPCRSACB31 | Restriction site optimized plasmid containing sacB and | pPCRSACB28 | (5) |
| | kanamycin resistance cassette. | | |
| pPCRUNKK37 | Expression vector to incorporate genes into A. | | (4) |
| | vinelandii behind Avin_16040 promoter. | | |
| pPCRUNKK41 | Optimized version of pPCRUNKK37 with additional | pPCRUNKK37 | This Study |
| | restriction sites and deoxyviolacein operon for | | |
| | purple/white selection. | | |
| pPCRURE2 | Plasmid containing flanking regions surrounding | | (6) |
| | ureABC genes from A. vinelandii. | | |
| pPCRURE90 | Moved lacZ and tetracycline selection cassette from | pPCRURE2, | This Study |
| | pLACZF19 into pPCRURE2. | pLACZF19 | |
| pPCRURE91 | Moved sacB and kanamycin selection cassette | pPCRURE2, | This Study |
| | pPCRSACB31 into pPCRURE2. | pPCRSACB31 | |

(2) Barney and Dietz, 2024. Microbial biotechnology 17(7): e14523.
(4) Barney and Plunkett, 2022. Appl Microbiol Biotechnol 106: 5051-5061.
(5) Dietz et al., 2024. Microbiology Spectrum 12(1): e0247823.
(6) Barney et al., 2015. Appl Environ Microbiol 81: 4316-4328
(7) Lenneman et al., 2013. Appl Environ Microbiol 79: 7055-7062.

TABLE 3
Primers used for the construction of Azotobacter vinelandii manipulated strains

| | | Plasmid |
|---|---|---|
| Primer | Sequence (all in 5′ to 3′ direction) | Construct |
| BBP2711 | NNNGGTACCA TATGAATACA AACCAAGATA ACGGAAATCA GCTCC | pPCRARG35-2 |
| | (SEQ ID NO: 1) | |
| BBP2712 | NNNAAGCTTA CAGCAGCTTC TTCCCTAACA GGGATTC (SEQ ID NO: 2) | pPCRARG35-2 |
| BBP3768 | NNNGAATTCG TTCCGAGTCG CTCTCCCGCT TTCACCGTC | pPCRAZP1 |
| | (SEQ ID NO: 3) | |
| BBP3769 | NNNAAGCTTG TGCGTCTGGC GCTGAAGGAT CTGTACCAG | pPCRAZP1 |
| | (SEQ ID NO: 4) | |
| BBP3770 | NNNGAATTCA CATCCATATG GACACTCGGC AGTTCGAAGCG | pPCRAZP4 |
| | CCAGTATTAT G (SEQ ID NO: 5) | |
| BBP3771 | NNNGAATTCA AGCTTGGATC CTGAGGCAAG GCAGCGCCGCT | pPCRAZP4 |
| | CGGCCCATCA C (SEQ ID NO: 6) | |
| BBP3927 | CATGCATAAT ACTGGCGCTT CGTTAAGGAG GTAAAACATA | pPCRAZP10 |
| | TGAAACATTC TTCCGA (SEQ ID NO: 7) | |
| BBP3928 | TCGGAAGAAT GTTTCATATG TTTTACCTCC TTAACGAAGC | pPCRAZP10 |
| | GCCAGTATTA TGCATG (SEQ ID NO: 8) | |
| BBP4015 | CTTAAATGTT GAGGTGGAAT CACATATGGA TAAAACGATT | pPCRARG44 |
| | TCGGTTATTG (SEQ ID NO: 9) | |
| BBP4016 | CAATAACCGA AATCGTTTTA TCCATATGTG ATTCCACCTC | pPCRARG44 |
| | AACATTTAAG (SEQ ID NO: 10) | |
| BBP5006 | CTCGAGCTTT GACTAAGGAG GTAAAAACAT ATGGATAAAAC | pPCRARG88 |
| | GATTTCGGTT ATTGGAATG (SEQ ID NO: 11) | |
| BBP5007 | CTGACGATTT GCCTGCTGGC GTTTTTTAAT GAC (SEQ ID NO: 12) | pPCRARG88 |
| BBP5016 | NNNTCTAGAC ATATGATGAA TCCATTAATT ATCAAACTGG | pPCRARG83 |
| | GCGGCGTAC (SEQ ID NO: 13) | |
| BBP5017 | NNNGAATTCT TAAGCTAAAA TCCGCGTACC CATCGGCATA CCG (SEQ | pPCRARG83 |
| | ID NO: 14) | |

### Urea Assays

Urea was quantified using a derivative of the previously described methods (Barney et al., 2015. Applied and Environmental Microbiology 81:4316-4328). Urea assay reagent was prepared by combining 2.94 grams of sulfuric acid, 0.34 grams of phosphoric acid, 2 mg of ferric chloride, 2 mg of thiosemicarbazide and 25 mg of diacetyl monoxime in 18 mL of water. Standards were prepared ranging from 0.166 mM to 1.66 mM, and 100 μL of standard or sample was added to a 2 mL microfuge tube, and 1 mL of assay reagent was added. Samples were incubated at 80° C. with occasional mixing by inverting for 20 minutes, then transferred to ice to stop the reaction. Samples were transferred to a cuvette and measured at 520 nm. Cultures of A. vinelandii strains were measured by removing 1 mL of culture volume and pelleting the cells at 10,000×g for one minute, then removing an aliquot of the supernatant for analysis.

### Culture of Algae for Nitrogen Delivery

To confirm that the enhanced urea production measured by urea assays could support a non-diazotrophic algal strain in co-culture, the green alga Chlorella sorokiniana (Arriola et al., 2017. The Plant Journal 93:566-586) was combined with various strains of A. vinelandii constructed here, and spotted onto plates of Bristol media lacking a nitrogen source, but supplemented with Burk's medium. Cells were grown on a light table with 150 μmol m⁻² s⁻¹ of photons and 14 hours of light followed by 10 hours of a dark light cycle for two weeks to screen for enhanced algal growth, similar to prior approaches (Barney et al., 2015. Applied and Environmental Microbiology 81:4316-4328; Dietz et al., 2024. Microbiology Spectrum 12).

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

What is claimed is:

1. A diazotrophic microbe genetically modified to increase production of extracellular urea compared to an unmodified version of the diazotrophic microbe.

2. The diazotrophic microbe of claim 1, wherein the diazotrophic microbe comprises a deletion or disruption of one or more genes of a urease operon.

3. The diazotrophic microbe of claim 1, wherein the diazotrophic microbe comprises one or more exogenous genes.

4. The diazotrophic microbe of claim 3, wherein at least one exogenous gene comprises E. coli arginine-insensitive acetylglutamate kinase (argB).

5. The diazotrophic microbe of claim 4, wherein expression of the E. coli argB is controlled by the promoter of conserved hypothetical protein (Avin_14720).

6. The diazotrophic microbe of claim 3, wherein at least one exogenous gene comprises B. subtilis arginine permease (rocE), B. subtilis arginase (argI), or both.

7. The diazotrophic microbe of claim 6, wherein expression of the B. subtilis arginine permease (rocE), B. subtilis arginase (argI), or both, is controlled by the promoter of S-layer protein (Avin_16040).

8. The diazotrophic microbe of claim 1, wherein the diazotrophic microbe is a species of the genus Azotobacter, a species of the genus Gluconacetobacter, a species of the genus Azoarcus, a species of the genus Azospirillum, a species of the genus Herbaspirillum, a species of the genus Klebsiella, a species of the genus Pseudomonas, or a species of the genus Paenibacillus.

9. The diazotrophic microbe of claim 8, wherein the diazotrophic microbe is A. vinelandii.

10. The diazotrophic microbe of claim 1, wherein the diazotrophic microbe produces at least 1.0 mM extracellular urea within 180 hours of culture in nitrogen-free medium.

11. The diazotrophic microbe of claim 1, wherein the diazotrophic microbe produces at least 5.0 mM extracellular urea within 180 hours of culture in nitrogen-free medium.

12. A co-culture comprising:
the diazotrophic microbe of claim 1; and
a second organism selected to feed on the extracellular urea provided by the diazotrophic microbe.

13. The co-culture of claim 12, further comprising culture medium.

14. The co-culture of claim 12, wherein the second organism is selected to produce a product of interest.

15. The co-culture of claim 12, wherein the second organism is a non-legume crop plant.

16. The co-culture of claim 15, wherein the non-legume crop plant is corn, wheat, rice, barley, or sugar beet.

17. A biofertilizer comprising the diazotrophic microbe of claim 1.

18. A method of increasing growth of a non-diazotrophic organism, the method comprising:
co-culturing the non-diazotrophic organism with the diazotrophic microbe of claim 1 in an amount effective to increase the growth of the non-diazotrophic organism compared to growth of the non-diazotrophic organism in the absence of the diazotrophic microbe.

19. The method of claim 18 wherein increasing the growth of the non-diazotrophic organism comprises increasing the growth rate of the non-diazotrophic organism.

20. The method of claim 18 wherein increasing the growth of the non-diazotrophic organism comprises increasing the cell density of the non-diazotrophic organism.

21. The method of claim 18 wherein increasing the growth of the non-diazotrophic organism comprises increasing the crop yield of the non-diazotrophic organism.


SEQUENCE LISTING (14 sequences)

SEQ ID NO: 1 - DNA, 45 bp, synthetic construct
  source 1..45: mol_type = other DNA
nnnggtaccatatgaatacaaaccaagataacggaaatcagctcc

SEQ ID NO: 2 - DNA, 37 bp, synthetic construct
  source 1..37: mol_type = other DNA
nnnaagcttacagcagcttcttccctaacagggattc

SEQ ID NO: 3 - DNA, 39 bp, synthetic construct
  source 1..39: mol_type = other DNA
nnngaattcgttccgagtcgctctcccgctttcaccgtc

SEQ ID NO: 4 - DNA, 39 bp, synthetic construct
  source 1..39: mol_type = other DNA
nnnaagcttgtgcgtctggcgctgaaggatctgtaccag

SEQ ID NO: 5 - DNA, 52 bp, synthetic construct
  source 1..52: mol_type = other DNA
nnngaattcacatccatatggacactcggcagttcgaagcgccagtattatg

SEQ ID NO: 6 - DNA, 52 bp, synthetic construct
  source 1..52: mol_type = other DNA
nnngaattcaagcttggatcctgaggcaaggcagcgccgctcggcccatcac

SEQ ID NO: 7 - DNA, 56 bp, synthetic construct
  source 1..56: mol_type = other DNA
catgcataatactggcgcttcgttaaggaggtaaaacatatgaaacattcttccga

SEQ ID NO: 8 - DNA, 56 bp, synthetic construct
  source 1..56: mol_type = other DNA
tcggaagaatgtttcatatgttttacctccttaacgaagcgccagtattatgcatg

SEQ ID NO: 9 - DNA, 50 bp, synthetic construct
  source 1..50: mol_type = other DNA
cttaaatgttgaggtggaatcacatatggataaaacgatttcggttattg

SEQ ID NO: 10 - DNA, 50 bp, synthetic construct
  source 1..50: mol_type = other DNA
caataaccgaaatcgttttatccatatgtgattccacctcaacatttaag

SEQ ID NO: 11 - DNA, 60 bp, synthetic construct
  source 1..60: mol_type = other DNA
ctcgagctttgactaaggaggtaaaaacatatggataaaacgatttcggttattggaatg

SEQ ID NO: 12 - DNA, 33 bp, synthetic construct
  source 1..33: mol_type = other DNA
ctgacgatttgcctgctggcgttttttaatgac

SEQ ID NO: 13 - DNA, 49 bp, synthetic construct
  source 1..49: mol_type = other DNA
nnntctagacatatgatgaatccattaattatcaaactgggcggcgtac

SEQ ID NO: 14 - DNA, 43 bp, synthetic construct
  source 1..43: mol_type = other DNA
nnngaattcttaagctaaaatccgcgtacccatcggcataccg